(19) United States
(12) Patent Application Publication
(10) Pub. No.: US 2020/0258231 A1
(43) Pub. Date: Aug. 13, 2020

(54) TUMOR TRACKING METHOD AND APPARATUS

(71) Applicant: OUR UNITED CORPORATION, Xi'an (CN)

(72) Inventors: Jiuliang LI, Xi'an (CN); Hao YAN, Xi'an (CN); Jinsheng LI, Xi'an (CN); Tianchang GOU, Xi'an (CN)

(21) Appl. No.: 16/865,148

(22) Filed: May 1, 2020

Related U.S. Application Data
(63) Continuation of application No. PCT/CN2017/109144, filed on Nov. 2, 2017.

(51) Int. Cl. G06T 7/00; G06T 3/60; G06T 7/73; G06T 19/20; A61N 5/10

(52) U.S. Cl. CPC G06T 7/0014; G06T 3/60; G06T 7/74; G06T 19/20; A61N 2005/1034; G06T 2207/30096; G06T 2219/2004; G06T 2210/41; A61N 5/1031

(57) ABSTRACT

A tumor tracking method is provided. The method includes: acquiring a tumor image at an N^{th} moment; determining a two-dimensional positional deviation of a tumor at the N^{th} moment according to the tumor image at the N^{th} moment and a tumor reference image corresponding to the tumor image at the N^{th} moment; determining a three-dimensional positional deviation of the tumor at the N^{th} moment according to the two-dimensional positional deviation of the tumor at the N^{th} moment and a predetermined two-dimensional positional deviation of the tumor at an (N−1)^{th} moment; and tracking the tumor according to the three-dimensional positional deviation of the tumor at the N^{th} moment.

### CROSS-REFERENCE TO RELATED APPLICATION

This application is a continuation application of International Application No.: PCT/CN2017/109144 filed on Nov. 2, 2017, the entire contents of which are incorporated herein by reference.

### TECHNICAL FIELD

The present disclosure relates to the field of radiotherapy, and more particularly, to a tumor tracking method and apparatus.

### BACKGROUND

Radiation therapy (referred to as a radiotherapy) is a treatment method for treating tumors by radioactive rays. The radiotherapy may cause apoptosis or necrosis of cancer cells, and is one of the major means for treating malignant tumors. The key technology of the radiotherapy is to maintain precise positioning of a tumor during the radiotherapy. However, in the process of actual radiotherapy, the patient's involuntary movement or the movement of patient's organs may cause movement of the tumor, thereby affecting the accuracy of positioning the radiotherapy target. Therefore, during the radiotherapy, it is generally necessary to perform tumor tracking to achieve precise positioning.

### SUMMARY

In a first aspect, a tumor tracking method is provided. The method is applied to a radiotherapy device including a tumor image acquisition apparatus and configured to acquire tumor images at different moments. The method includes:

acquiring a tumor image at an N^{th} moment, wherein N=2, 3, 4 . . . M, M being a positive integer;

determining a two-dimensional positional deviation of the tumor at the N^{th} moment according to the tumor image at the N^{th} moment and a tumor reference image corresponding to the tumor image at the N^{th} moment;

determining a three-dimensional positional deviation of the tumor at the N^{th} moment according to the two-dimensional positional deviation of the tumor at the N^{th} moment and a predetermined two-dimensional positional deviation of the tumor at an (N−1)^{th} moment, wherein the two-dimensional positional deviation of the tumor at the (N−1)^{th} moment is a two-dimensional positional deviation determined according to a tumor image at the (N−1)^{th} moment and a tumor reference image corresponding to the tumor image at the (N−1)^{th} moment, and a position at which the tumor image at the N^{th} moment is acquired is different from a position at which the tumor image at the (N−1)^{th} moment is acquired; and

tracking the tumor according to the three-dimensional positional deviation of the tumor at the N^{th} moment.

In a second aspect, a tumor tracking method is provided. The method includes:

acquiring a first image of a tumor at a first moment;

acquiring a second image of the tumor at a second moment; and

determining a position of the tumor based on the first image and the second image;

wherein the first moment and the second moment are adjacent moments, and the first image and the second image have different image planes.

In a third aspect, a tumor tracking apparatus is provided. The apparatus includes a processor, configured to:

acquire a first image of a tumor at a first moment;

acquire a second image of the tumor at a second moment; and

determine a position of the tumor based on the first image and the second image;

wherein the first moment and the second moment are adjacent moments, and the first image and the second image have different image planes.

In a fourth aspect, a computer readable storage medium is provided. The computer readable storage medium is configured to store at least one instruction therein, which, when running on a processing component of a computer, causes the processing component to perform the tumor tracking method in the first aspect.

### BRIEF DESCRIPTION OF THE DRAWINGS

FIG. 1 is a diagram of an application scenario of a radiotherapy device according to an embodiment of the present disclosure;

FIG. 2 is a schematic diagram of acquisition of a tumor image according to an embodiment of the present disclosure;

FIG. 3 is a flowchart of a tumor tracking method according to an embodiment of the present disclosure;

FIG. 4 is a flowchart of a method for determining a three-dimensional positional deviation of a tumor according to an embodiment of the present disclosure;

FIG. 5 is a flowchart of a method for tracking the tumor according to the three-dimensional positional deviation of the tumor according to an embodiment of the present disclosure;

FIG. 6 is a flowchart of another tumor tracking method according to an embodiment of the present disclosure;

FIG. 7 is a flowchart of still another tumor tracking method according to an embodiment of the present disclosure;

FIG. 8 is a schematic diagram of acquisition of a tumor image is acquired in an arc treatment mode according to an embodiment of the present disclosure;

FIG. 9 is a flowchart of a method for tracking the tumor in the arc treatment mode according to an embodiment of the present disclosure;

FIG. 10 is a schematic diagram of acquisition of a tumor image in a fixed-point treatment mode according to an embodiment of the present disclosure;

FIG. 11 is a flowchart of a method for tracking the tumor in the fixed-point treatment mode according to an embodiment of the present disclosure; and

FIG. 12 is a schematic structural diagram of a radiotherapy system according to an embodiment of the present disclosure.

### DETAILED DESCRIPTION

In the related art, a radiation therapy device (referred to as a radiotherapy device) is usually used to track a tumor. The radiotherapy device includes a rotation frame, a treatment head, a control component, and two imaging systems. Each imaging system includes an X-ray tube and a flat panel detector. The rotation frame is in a cylindrical structure. The treatment head, the X-ray tube, and the flat panel detector are all arranged on the rotation frame, and the position of the X-ray tube of each imaging system on the rotation frame faces the position of the flat panel detector on the rotation frame. A central angle corresponding to a circular arc between the two X-ray tubes is 90 degrees. The control component is connected to the rotation frame, the treatment head, the X-ray tubes and the flat panel detectors respectively. During the tumor tracking process, a patient is positioned in the rotation frame by a treatment bed. The control component controls the rotation frame to rotate, and controls the two X-ray tubes to emit X-rays to the tumor simultaneously and periodically. The X-rays emitted by each X-ray tube pass through the tumor to the corresponding flat panel detector. The control component determines a two-dimensional image according to the X-rays received by each flat panel detector to obtain two two-dimensional images, and determines a three-dimensional position of the tumor according to the two two-dimensional images, thereby realizing tumor tracking.

In the process of implementing the present disclosure, the inventors have found that the related art has at least the following problems:

In the related art, two imaging systems are needed for tumor tracking, which impose stricter requirements on a space and cable layout for a radiotherapy device, and the flat panel detectors are relatively expensive. Therefore, a device for tumor tracking in the related art is highly complex and costly.

For clearer descriptions of the principles, technical solutions and advantages in the embodiments of the present disclosure, the present disclosure is described in detail below in combination with the accompanying drawings. Apparently, the described embodiments are merely some embodiments, rather than all embodiments, of the present disclosure.

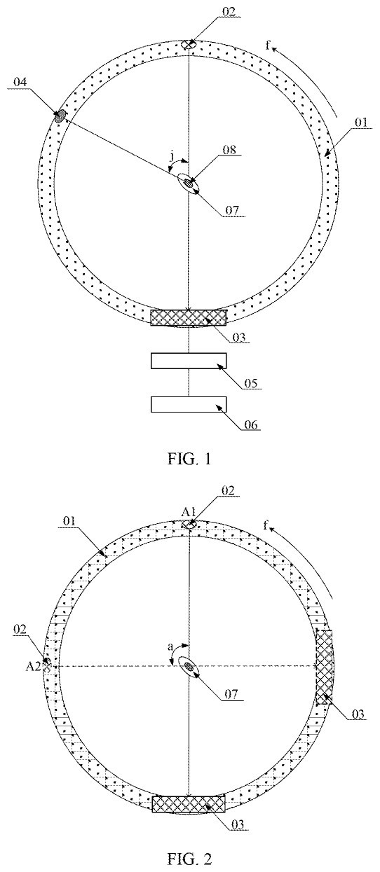

FIG. 1 is a diagram of an application scene of a radiotherapy device according to an embodiment of the present disclosure. Referring to FIG. 1, the radiotherapy device includes: a rotation frame 01, an imaging source 02, a detector 03, a treatment head 04, a processing component 05, and a control component 06. The imaging source 02 may be an X-ray tube, and the detector 03 may be a flat panel detector. The imaging source 02 and the detector 03 may form an imaging system. The imaging system may also be referred to as a tumor image acquisition apparatus.

The rotation frame 01 may be in a cylindrical structure, which may specifically be a drum. The tumor image acquisition apparatus and the treatment head 04 are respectively arranged on the rotation frame 01, and are arranged on the same circumference of the rotation frame 01. A position of the imaging source 02 on the rotation frame 01 faces a position of the detector 03 on the rotation frame 01, such that rays emitted by the imaging source 02 may be received by the detector 03. A central angle corresponding to a circular arc between the treatment head 04 and the imaging source 02 may be j. In this embodiment of the present disclosure, the rotation frame 01 may rotate in a rotation direction f, and drive the tumor image acquisition apparatus and the treatment head 04 to rotate.

The processing component 05 may be connected to the detector 03. The control component 06 is respectively connected to the rotation frame 01, the imaging source 02 and the treatment head 04, and also connected to the control component 06. The control component 06 may control the rotation frame 01 to rotate in the rotation direction f, and control the imaging source 02 and the treatment head 04 to emit rays. The detector 03 may receive the rays emitted by the imaging source 02. The processing component 05 may determine the tumor images based on the rays received by the detector 03. The processing component 05 may be configured in a computer, and the processing component 05 may be a processor of the computer. Alternatively, the processing component 05 may be processing software, and the control component 06 may be a controller.

It should be noted that, in practice, the rotation frame 01 may also be a cantilever or a mechanical arm, which may drive the tumor image acquisition apparatus and the treatment head 04 to rotate circumferentially. The processing component 05 and the control component 06 may be implemented as a whole, or may be provided separately. For specific structures of the rotation frame 01, the imaging source 02, the detector 03 and the treatment head 04, reference may be made to the related arts, and are not repeated in the embodiments of the present disclosure.

As shown in FIG. 1, a tumor 08 grows in the body of a patient 07. If the tumor is a lung tumor, the tumor 08 is moveable regularly with the breathes of the patient 07. In the case that the radiotherapy device is used, the patient 07 is positioned in the rotation frame 01 through a treatment bed (not shown in FIG. 1). The patient is asked to keep a body position fixed on the treatment bed and maintain smooth breathing. Next, the control component 06 controls the rotation frame 01 to rotate in the rotation direction f. During the rotation of the rotation frame 01:

in conjunction with FIG. 1 and FIG. 2, at an (N−1)^{th} moment, the control component 06 controls the tumor image acquisition apparatus to acquire a tumor image at the (N−1)^{th} moment. The processing component 05 determines a two-dimensional positional deviation of the tumor at the (N−1)^{th} moment according to the tumor image at the (N−1)^{th} moment and a tumor reference image corresponding to the tumor image at the (N−1)^{th} moment. At the (N−1)^{th} moment, the imaging source 02 may be located at a position point A1 or a position point A2, or the imaging source 02 may also be located at another position point (not shown in FIG. 2). If the imaging source 02 at the (N−1)^{th} moment is located at the position point A1, the tumor reference image corresponding to the tumor image at the (N−1)^{th} moment is also a tumor reference image corresponding to the position point A1. If the imaging source 02 at the (N−1)^{th} moment is located at the position point A2, the tumor reference image corresponding to the tumor image at the (N−1)^{th} moment is also a tumor reference image corresponding to the position point A2. In this embodiment of the present disclosure, the process that the control component 06 controls the tumor image acquisition apparatus to acquire the tumor images be as follows: the control component 06 controls the imaging source 02 to emit rays to the tumor, and the rays pass through the tumor to the detector 03; the detector 03 receives the rays passing through the tumor; and the processing component 05 determines the tumor images according to the rays received by the detector 03. The process that the processing component 05 determines the two-dimensional positional deviation of the tumor at the (N−1)^{th} moment according to the tumor image at the (N−1)^{th} moment and the tumor reference image corresponding to the tumor image at the (N−1)^{th} moment may include: the processing component 05 compares the tumor image at the (N−1)^{th} moment with the tumor reference image corresponding to the tumor image at the (N−1)^{th} moment to determine the two-dimensional positional deviation of the tumor at the (N−1)^{th} moment.

In conjunction with FIG. 1 and FIG. 2, at an N^{th} moment, the control component 06 controls the tumor image acquisition apparatus to acquire a tumor image at the N^{th} moment. The processing component 05 determines a two-dimensional positional deviation of the tumor at the N^{th} moment according to the tumor image at the N^{th} moment and a tumor reference image corresponding to the tumor image at the N^{th} moment. At the N^{th} moment, the imaging source 02 may be located at a position point A1 or a position point A2, or the imaging source 02 may also be located at another position point (not shown in FIG. 2). In addition, the position point at which the imaging source 02 is located at the N^{th} moment is different from the position point at which the imaging source 02 is located at the (N−1)^{th} moment. If the imaging source 02 at the N^{th} moment is located at the position point A1, the tumor reference image corresponding to the tumor image at the N^{th} moment is also a tumor reference image corresponding to the position point A1. If the imaging source 02 at the N^{th} moment is located at the position point A2, the tumor reference image corresponding to the tumor image at the N^{th} moment is also a tumor reference image corresponding to the position point A2. The process that the control component 06 controls the tumor image acquisition apparatus to acquire the tumor images and the process that the processing component 05 determines the two-dimensional positional deviation of the tumor at the N^{th} moment according to the tumor image at the N^{th} moment and the tumor reference image corresponding to the tumor image at the N^{th} moment are the same as or similar to the process related to the above-mentioned (N−1)^{th} moment, and are not repeated herein.

After determining the two-dimensional positional deviation of the tumor at the (N−1)^{th} moment and the two-dimensional positional deviation of the tumor at the N^{th} moment, the processing component 05 may determine a three-dimensional positional deviation of the tumor at the N^{th} moment according to the two-dimensional positional deviation of the tumor at the (N−1)^{th} moment and the two-dimensional positional deviation of the tumor at the N^{th} moment. The control component 06 tracks the tumor according to the three-dimensional positional deviation of the tumor at the N^{th} moment. Optionally, the process that the processing component 05 determines the three-dimensional positional deviation of the tumor at the N^{th} moment according to the two-dimensional positional deviation of the tumor at the (N−1)^{th} moment and the two-dimensional positional deviation of the tumor at the N^{th} moment may be as follows: the processing component 05 calculates the three-dimensional positional deviation of the tumor at the N^{th} moment in conjunction with the two-dimensional positional deviation of the tumor at the (N−1)^{th} moment and the two-dimensional positional deviation of the tumor at the N^{th} moment. The process that the control component 06 tracks the tumor according to the three-dimensional positional deviation of the tumor at the N^{th} moment may be as follows: the control component 06 tracks the tumor according to a relationship between the three-dimensional positional deviation of the tumor at the N^{th} moment and a predetermined deviation range, and may be specifically as follows: the control component 06 automatically corrects the position of the tumor if the three-dimensional positional deviation of the tumor at the N^{th} moment falls within the predetermined deviation range; the control component 06 performs an alarm operation to make a prompt of manually correcting the position of the tumor if the three-dimensional positional deviation of the tumor at the N^{th} moment is greater than an upper limit of the predetermined deviation range; and the position of the tumor does not need to be corrected if the three-dimensional positional deviation of the tumor at the N^{th} moment is smaller than a lower limit of the predetermined deviation range. The process that the control component 06 automatically corrects the position of the tumor may include: the control component 06 controls a treatment bed of the radiotherapy device to move according to the three-dimensional positional deviation of the tumor at the N^{th} moment, such that the tumor coincides with a focal point of the radiotherapy device; or the control component 06 adjusts a multi-leaf collimator of the radiotherapy device according to the three-dimensional positional deviation of the tumor at the N^{th} moment, such that a radiation field of the multi-leaf collimator coincides with the tumor.

In this embodiment of the present disclosure, an included angle between the position at which the tumor image at the N^{th} moment is acquired and the position at which the tumor image at the (N−1)^{th} moment is acquired may be defined according to the actual needs. Optionally, the included angle between the position at which the tumor image at the N^{th} moment is acquired and the position at which the tumor image at the (N−1)^{th} moment is acquired may be within a value range of 45-135 degrees. Optionally, as shown in FIG. 2, a central angle corresponding to a circular arc between the position point A2 and the position point A1 may be a, and the included angle between the position at which the tumor image at the N^{th} moment is acquired and the position at which the tumor image at the (N−1)^{th} moment is acquired may be the central angle a. Therefore, the central angle a may be 45-135 degrees. That is, the central angle a is within the value range of 45-135 degrees, for example, a=90 degrees.

In this embodiment of the present disclosure, the detector 03 may include an analog-to-digital converter (ADC). The process that the processing component 05 determines the tumor images according to the rays received by the detector 03 may include: the detector 03 converts the received rays into optical signals, and then converts the optical signals into analog signals; the ADC converts the analog signals into digital signals and sends the digital signals to the processing component 05; and the processing component 05 generates the tumor images according to the received digital signals.

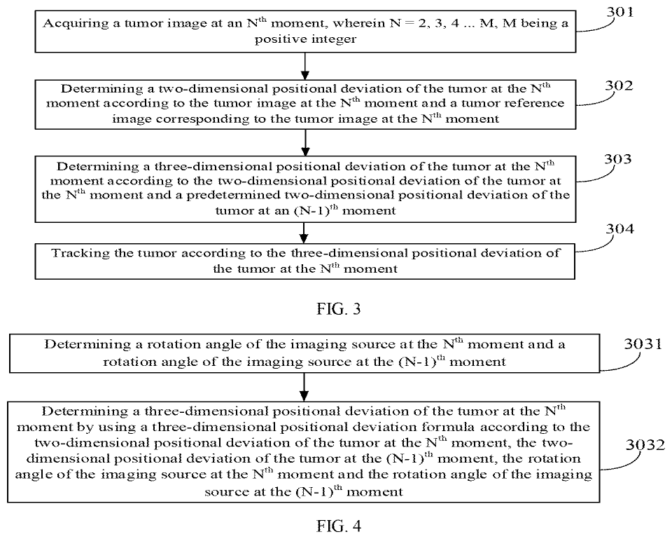

FIG. 3 is a flowchart of a tumor tracking method according to an embodiment of the present disclosure. This embodiment is hereinafter illustrated by applying the tumor tracking method to the radiotherapy device shown in FIG. 1. The tumor tracking method may be performed by a tumor tracking apparatus which includes a tumor image acquisition apparatus (including an imaging source and a detector) in the radiotherapy device as shown in FIG. 1, a processing component, a control component, and the like. Referring to FIG. 3, the tumor tracking method includes the following steps.

In step 301, a tumor image at an N^{th} moment is acquired, wherein N is 2, 3, 4 . . . M, M being a positive integer.

In this embodiment, the tumor image at an N^{th} moment is acquired by the tumor image acquisition apparatus. The tumor image acquisition apparatus may include an imaging source and a detector. The tumor image acquisition apparatus may rotate around the circumference of the tumor at a uniform speed or at a non-uniform speed. At the N^{th} moment, the imaging source may emit rays to the tumor. The rays pass through the tumor to the detector and are received by the detector. The tumor image determined by the processing component according to the rays received by the detector is the tumor image at the N^{th} moment. The rays may be X-rays. N is 2, 3, 4 . . . M, M being a positive integer. That is, N is a positive integer greater than or equal to 2.

Exemplarily, as shown in FIG. 2, it is assumed that the imaging source 02 rotates to the position point A2 at the N^{th} moment, the imaging source 02 emits rays from the position point A2 shown in FIG. 2 to the tumor in the body of the patient 07. The rays pass through the tumor in the body of the patient 07 to the detector 03 and are received by the detector 03. The tumor image determined by the processing component according to the rays received by the detector 03 is the tumor image at the N^{th} moment.

In step 302, a two-dimensional positional deviation of the tumor at the N^{th} moment is determined according to the tumor image at the N^{th} moment and a tumor reference image corresponding to the tumor image at the N^{th} moment.

The tumor reference image corresponding to the tumor image at the N^{th} moment is also the tumor reference image corresponding to the position point at which the imaging source is located at the N^{th} moment. In this embodiment of the present disclosure, the processing component may store a predetermined image library therein. The predetermined image library includes tumor reference images corresponding to respective position points of a plurality of position points. The tumor reference image corresponding to each position point is a tumor reference image determined based on an optical signal emitted from the corresponding position point to the tumor.

The processing component may first acquire a tumor reference image corresponding to a position point at which the imaging source is located at the N^{th} moment from the predetermined image library, determine the tumor reference image as the tumor reference image corresponding to the tumor image at the N^{th} moment, then compare the tumor image at the N^{th} moment with the tumor reference image corresponding to the tumor image at the N^{th} moment, and determine a two-dimensional positional deviation of the tumor at the N^{th} moment. The two-dimensional positional deviation of the tumor at the N^{th} moment may include a joint positional deviation U_{N} of the tumor at the N^{th} moment in an x-axis direction and in a z-axis direction, and a positional deviation Y_{N} of the tumor at the N^{th} moment in a y-axis direction. An origin of the x-axis, y-axis and z-axis is a midpoint of a connecting line between the imaging source and the detector. The radiotherapy device may include a treatment bed. The y-axis is parallel to a length direction of the treatment bed of the radiotherapy device. The x-axis is in the same plane with the y-axis and perpendicular to the y-axis. The z-axis is perpendicular to the plane composed of the x-axis and the y-axis.

It should be noted that, in this embodiment of the present disclosure, the tumor reference image in the predetermined image library may be a pre-acquired electronic computed tomography (CT) digitally reconstructed two-dimensional image of the tumor. Therefore, the tumor reference image corresponding to the tumor image at the N^{th} moment may be a pre-acquired CT image of the tumor. The predetermined image library may be formed before the treatment or may be formed during the treatment. If the predetermined image library is formed before the treatment, the tumor may be scanned with a CT machine before the treatment to obtain a CT image sequence of the tumor. The CT image sequence includes a series of CT images. A CT digitally reconstructed two-dimensional image corresponding to each of a plurality of position points is obtained by calculation with an image reconstruction algorithm. The CT digitally reconstructed two-dimensional image is also the tumor reference image. If the predetermined image library is formed during the treatment, the tumor may be scanned with the CT machine before the treatment to obtain a CT image sequence of the tumor. The CT image sequence includes a series of CT images. During the treatment, in the case that the two-dimensional positional deviation of the tumor at the N^{th} moment needs to be determined, a CT digitally reconstructed two-dimensional image corresponding to the tumor image at the N^{th} moment is obtained by calculation with the image reconstruction algorithm. The CT digitally reconstructed two-dimensional image is also the tumor reference image corresponding to the tumor image at the N″ moment. It should be noted that this embodiment of the present disclosure is described by taking a case of scanning the tumor with the CT machine and acquiring a tumor reference image as an example. In practice, the tumor reference image may also be acquired by magnetic resonance imaging (MRI), which is not repeated in this embodiment of the present disclosure.

In step 303, a three-dimensional positional deviation of the tumor at the N^{th} moment is determined according to the two-dimensional positional deviation of the tumor at the N^{th} moment and a predetermined two-dimensional positional deviation of the tumor at the (N−1)^{th} moment.

The two-dimensional positional deviation of the tumor at the (N−1)^{th} moment is a two-dimensional positional deviation determined according to the tumor image at the (N−1)^{th} moment and the tumor reference image corresponding to the tumor image at the (N−1)^{th} moment, and a position at which the tumor image at the N^{th} moment is acquired is different from a position at which the tumor image at the (N−1)^{th} moment is acquired. The process that the processing component determines the two-dimensional positional deviation of the tumor at the (N−1)^{th} moment is similar to the process that the processing component determines the two-dimensional positional deviation of the tumor at the N^{th} moment in steps 301 to 302, which is not repeated in this embodiment. It should be noted that the position at which the tumor image at the N^{th} moment is acquired is different from the position at which the tumor image at the (N−1)^{th} moment is acquired. For example, when a position point at which the tumor image at the N^{th} moment is acquired is the position point A2 in FIG. 2, a position point at which the tumor image at the (N−1)^{th} moment is acquired is the position point A1 in FIG. 2. In addition, similar to the two-dimensional positional deviation of the tumor at the N^{th} moment, the two-dimensional positional deviation of the tumor at the (N−1)^{th} moment includes a joint positional deviation U_{N-1} of the tumor at the (N−1)^{th} moment in the x-axis direction and in the z-axis direction, and a positional deviation Y_{N-1} of the tumor at the (N−1)^{th} moment in the y-axis direction. Optionally, the included angle between the position at which the tumor image at the N^{th} moment is acquired and the position at which the tumor image at the (N−1)^{th} moment is acquired may be within the value range of 45-135 degrees. For example, as shown in FIG. 2, an included angle between the position point A2 and the position point A1 may be within the value range of 45-135 degrees (that is, the central angle a corresponding to the arc formed between the position point A2 and the position point A1 is within the value range of 45-135 degrees).

In step 304, the tumor is tracked according to the three-dimensional positional deviation of the tumor at the N^{th} moment.

The control component tracks the tumor according to a relationship between the three-dimensional positional deviation of the tumor at the N^{th} moment and a predetermined deviation range.

In summary, the tumor tracking method includes: acquiring the tumor image at the N^{th} moment; determining the two-dimensional positional deviation of the tumor at the N^{th} moment according to the tumor image at the N^{th} moment and the tumor reference image corresponding to the tumor image at the N^{th} moment; determining the three-dimensional positional deviation of the tumor at the N^{th} moment according to the two-dimensional positional deviation of the tumor at the N^{th} moment and the predetermined two-dimensional positional deviation of the tumor at the (N−1)^{th} moment; and tracking the tumor according to the three-dimensional positional deviation of the tumor at the N^{th} moment. Since the tumor tracking may be achieved just by adopting the tumor image acquisition apparatus (that is, an imaging system, which includes an imaging source and a detector), the present disclosure solves the problem that a tumor tracking device is highly complex and costly, and contributes to reducing the complexity and the cost of the tumor tracking device.

Step 303 is described hereinafter. FIG. 4 illustrates a flowchart of a method for determining the three-dimensional positional deviation of the tumor at the N^{th} moment according to the two-dimensional positional deviation of the tumor at the N^{th} moment and the two-dimensional positional deviation of the tumor at the (N−1)^{th} moment. Referring to FIG. 4, the method includes the following sub-steps.

In sub-step 3031, a rotation angle of the imaging source at the N^{th} moment and a rotation angle of the imaging source at the (N−1)^{th} moment are determined.

During the entire tumor tracking process, the rotation frame may rotate to drive the imaging source to rotate circumferentially. During the rotation of the imaging source, the rotation angles of the imaging source at different moments may be determined by a rotation frame driving apparatus or an encoder. The rotation angles of the imaging source at different moments may include the rotation angle of the imaging source at the N^{th} moment and the rotation angle of the imaging source at the (N−1)^{th} moment. The rotation angle of the imaging source at the N^{th} moment may be a rotation angle of the imaging source from a moment at which the imaging source begins to rotate around the tumor to the N^{th} moment. The rotation angle of the imaging source at the (N−1)^{th} moment may be a rotation angle of the imaging source from a moment at which the imaging source begins to rotate around the tumor to the (N−1)^{th} moment.

In this embodiment of the present disclosure, the rotation angle of the imaging source that begins to rotate around the tumor may be determined to be 0 degree. An included angle between the position point at which the imaging source is located at the (N−1)^{th} moment and the position point at which the imaging source that begins to rotate around the tumor is located (that is, a central angle corresponding to a circular arc between the position point at which the imaging source is located at the N^{th} moment and the position point at which the imaging source that begins to rotate around the tumor is located) is determined as the rotation angle of the imaging source at the N^{th} moment. An included angle between the position point at which the imaging source is located at the (N−1)^{th} moment and the position point at which the imaging source that begins to rotate around the tumor is located (that is, a central angle corresponding to a circular arc between the position point at which the imaging source is located at the (N−1)^{th} moment and the position point at which the imaging source that begins to rotate around the tumor is located) is determined as the rotation angle of the imaging source at the (N−1)^{th} moment. Alternatively, when the imaging source rotates around the tumor at a constant speed, an N^{th} time difference between the N^{th} moment and the moment at which the imaging source begins to rotate around the tumor, and an (N−1)^{th} time difference between the (N−1)^{th} moment and the moment at which the imaging source begins to rotate around the tumor may be determined. A rotation speed of the imaging source rotating around the circumference of the tumor at a uniform speed is determined. A product of the N^{th} time difference and the rotation speed is determined as the rotation angle of the imaging source at the N^{th} moment. A product of the (N−1)^{th} time difference and the rotation speed is determined as the rotation angle of the imaging source at the (N−1)^{th} moment.

In step 3032, a three-dimensional positional deviation of the tumor at the N^{th} moment is determined according to the two-dimensional positional deviation of the tumor at the N^{th} moment, the two-dimensional positional deviation of the tumor at the (N−1)^{th} moment, the rotation angle of the imaging source at the N^{th} moment and the rotation angle of the imaging source at the (N−1)^{th} moment.

In this embodiment of the present disclosure, a three-dimensional positional deviation formula may be:

X=(U_{N}×Sin R_{N-1}U_{N-1}×Sin R_{N})/(Cos R_{N-1}×Sin R_{N}−Cos R_{N}×Sin R_{N-1});

Y=Y_{N};

Z=(U_{N}×Cos R_{N-1}−U_{N-1}×Cos R_{N})/(Sin R_{N-1}×Cos R_{N}−Sin R_{N}×Cos R_{N-1});

in which, X is the positional deviation of the tumor in the x-axis direction; Y is the positional deviation of the tumor in the y-axis direction; Z is the positional deviation of the tumor in the z-axis direction; an origin of the x-axis, the y-axis and the z-axis is a midpoint of the connecting line between the imaging source and the detector, and this midpoint may also be referred to as a treatment isocenter; the y-axis is parallel to the lengthwise direction of the treatment bed of the radiotherapy device, wherein when the patient is lying on the treatment bed, a longitudinal direction of the patient may be parallel to the lengthwise direction of the treatment bed, the x-axis is in the same plane with the y-axis and perpendicular to the y-axis, and the z axis is perpendicular to the plane composed of the x axis and the y axis; U_{N} is the joint positional deviation of the tumor at the N^{th} moment in the x-axis direction and in the z-axis direction; R_{N} is the rotation angle of the imaging source at the N^{th} moment; Y_{N} is the positional deviation of the tumor at the N^{th} moment in the y-axis direction; U_{N-1} is the joint positional deviation of the tumor at the (N−1)^{th} moment in the x-axis direction and in the z-axis direction; and R_{N-1} is the rotation angle of the imaging source at the (N−1)^{th} moment. Sin R_{N} represents a sine value of R_{N}; Sin R_{N-1} represents a sine value of R_{N-1}; Cos R_{N} represents a cosine value of R_{N}; Cos R_{N-1} represents a cosine value of R_{N-1}; and/represents a division sign. The relationships of U_{N}, U_{N-1}, R_{N}, R_{N-1}, X, Y, and Z may be U_{N}=X×Cos R_{N}+Z×Sin R_{N}, U_{N-1}=X×Cos R_{N-1}+Z×Sin R_{N-1}.

Optionally, the processing component may calculate the positional deviation of the tumor at the N^{th} moment in the x-axis direction, the positional deviation of the tumor at the N^{th} moment in the y-axis direction and the positional deviation of the tumor at the N^{th} moment in the z-axis direction by substituting the joint positional deviation U_{N} of the tumor at the N^{th} moment in the x-axis direction and in the z-axis direction, the joint positional deviation U_{N-1} of the tumor at the (N−1)^{th} moment in the x-axis direction and in the z-axis direction, the rotation angle R_{N} of the imaging source at the N^{th} moment, the rotation angle R_{N-1} of the imaging source at the (N−1)^{th} moment and the positional deviation Y_{N} of the tumor at the N^{th} moment in the y-axis direction into the above three-dimensional positional deviation formula, thereby obtaining the three-dimensional positional deviation of the tumor at the N^{th} moment.

It should be noted that in the case that the three-dimensional positional deviation of the tumor at the N^{th} moment is determined according to the two-dimensional positional deviation of the tumor at the N^{th} moment and the two-dimensional positional deviation of the tumor at the (N−1)^{th} moment, Y=Y_{N}, wherein Y_{N} is the positional deviation of the tumor at the N^{th} moment in the y-axis direction, and the N^{th} moment is later than the (N−1)^{th} moment. In practice, when the three-dimensional positional deviation of the tumor is calculated, Y is a most recently acquired positional deviation of the tumor in the y-axis direction. For example, when the three-dimensional positional deviation of the tumor at the (N−1)^{th} moment is determined according to the two-dimensional positional deviation of the tumor at the (N−1)^{th} moment and a two-dimensional positional deviation of the tumor at an (N−2)^{th} moment. Y=Y_{N-1}, wherein Y_{N-1} is the positional deviation of the tumor at the (N−1)^{th} moment in the y-axis direction, and the (N−1)^{th} moment is later than the (N−2)^{th} moment. When a three-dimensional positional deviation of the tumor at an (N+1)^{th} moment is determined according to a two-dimensional positional deviation of the tumor at the (N+1)^{th} moment and the two-dimensional positional deviation of the tumor at the N^{th} moment, Y=Y_{N}+1, wherein Y_{N}+1 is the positional deviation of the tumor at the (N+1)^{th} moment in the y-axis direction, and the (N+1)^{th} moment is later than the N^{th} moment, and so on. That is, when the three-dimensional positional deviation of the tumor is determined according to two two-dimensional positional deviations obtained from the tumor images acquired for two consecutive times, the Y in the three-dimensional positional deviation is equal to a positional deviation of the tumor in the finally acquired two-dimensional positional deviation in the y-axis direction among the two two-dimensional positional deviations.

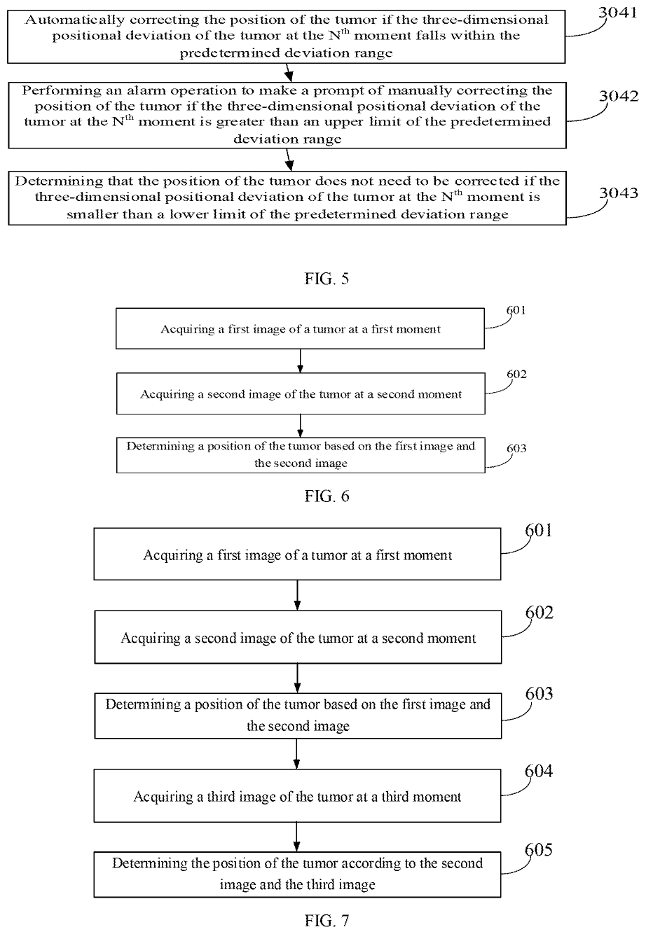

Step 304 is be described hereinafter. FIG. 5 illustrates a flowchart of a method for tracking the tumor according to the relationship of the three-dimensional positional deviation of the tumor at the N^{th} moment and the predetermined deviation range according to an embodiment of the present disclosure. Referring to FIG. 5, the method includes the following sub-steps.

In sub-step 3041, the position of the tumor is automatically corrected if the three-dimensional positional deviation of the tumor at the N^{th} moment falls within the predetermined deviation range.

In this embodiment, the control component may compare the three-dimensional positional deviation of the tumor at the N^{th} moment with the predetermined deviation range to determine whether the three-dimensional positional deviation of the tumor at the N^{th} moment falls within the predetermined deviation range. If the three-dimensional positional deviation of the tumor at the N^{th} moment falls within the predetermined deviation range, the control component automatically corrects the position of the tumor.

Optionally, the predetermined deviation range may be a deviation range of a square root of the three-dimensional positional deviation. The processing component calculates the square root of the three-dimensional positional deviation of the tumor at the N″ moment according to a square root formula, and then compares the square root of the three-dimensional positional deviation of the tumor at the N^{th} moment with an upper limit of the predetermined deviation range and a lower limit of the predetermined deviation range to determine whether the square root of the three-dimensional positional deviation of the tumor at the N″ moment falls within the predetermined deviation range. If the square root of the three-dimensional positional deviation of the tumor at the N^{th} moment is smaller than the upper limit of the predetermined deviation range and greater than the lower limit of the predetermined deviation range, or, if the square root of the three-dimensional positional deviation of the tumor at the N″ moment is equal to the upper limit of the predetermined deviation range, or, if the square root of the three-dimensional positional deviation of the tumor at the N^{th} moment is equal to the lower limit of the predetermined deviation range, the processing component determines that the square root of the three-dimensional positional deviation of the tumor at the N^{th} moment falls within the predetermined deviation range. The square root formula may be: d=\sqrt{X²+Y²+Z²}, in which X is the positional deviation of the tumor in the x-axis direction, Y is the positional deviation of the tumor in the y-axis direction, Z is the positional deviation of the tumor in the z-axis direction, and d is the square root of the three-dimensional positional deviation. The processing component may calculate the square root of the three-dimensional positional deviation of the tumor at the N^{th} moment by substituting the X value, the Y value and the Z value of the three-dimensional positional deviation of the tumor at the N^{th} moment calculated in step 303 into the square root formula.

In this embodiment, the radiotherapy device may be a multi-source focused radiotherapy device or a conformal intensity-modulated radiotherapy device. The multi-source focused radiotherapy device is provided with a treatment head loaded with a plurality of radioactive sources, and radioactive rays emitted by the plurality of radioactive sources may be converged at one point. The convergence point of the radioactive rays emitted by the plurality of radioactive sources may be referred to as a focal point of the multi-source focused radiotherapy device. If the radiotherapy device is the multi-source focused radiotherapy device, the control component may move the treatment bed of the radiotherapy device according to the three-dimensional positional deviation of the tumor at the N^{th} moment, such that the tumor coincides with the focal point of the multi-source focused radiotherapy device, thereby correcting the position of the tumor. The conformal intensity-modulated radiotherapy device generally has one treatment head, and also includes a multi-leaf collimator (MLC) which cooperates with the treatment head and has a radiation field, wherein radioactive rays emitted by the treatment head may be irradiated to the tumor through the irradiation field of the multi-leaf collimator. If the radiotherapy device is the conformal intensity-modulated radiotherapy device, the control component may adjust the multi-leaf collimator of the radiotherapy device according to the three-dimensional positional deviation of the tumor at the N^{th} moment, such that the irradiation field of the multi-leaf collimator coincides with the tumor, thereby correcting the position of the tumor.

In sub-step 3042, an alarm operation is performed to make a prompt of manually correcting the position of the tumor if the three-dimensional positional deviation of the tumor at the N^{th} moment is greater than an upper limit of the predetermined deviation range.

The control component may compare the three-dimensional positional deviation of the tumor at the N^{th} moment with the upper limit of the predetermined deviation range to determine whether the three-dimensional positional deviation of the tumor at the N^{th} moment is greater than the upper limit of the predetermined deviation range. If the three-dimensional positional deviation of the tumor at the N^{th} moment is greater than the upper limit of the predetermined deviation range, it indicates that the positional deviation of the tumor is relatively large. In this case, the position of the tumor cannot be corrected no matter how the radiotherapy device is adjusted. Therefore, the control component may perform an alarm operation to make a prompt of manually correcting the position of the tumor. Optionally, an alarm prompt sound may be sent, or prompt information may be generated and displayed, or the like. After the control component performs the alarm operation, the staff may manually correct the position of the tumor according to the alarm prompt. For example, the patient's position on the treatment bed is re-fixed, or the like.

Optionally, the predetermined deviation range may be a deviation range of a square root of the three-dimensional positional deviation. The control component may calculate the square root of the three-dimensional positional deviation of the tumor at the N^{th} moment according to the square root formula, and then compare the square root of the three-dimensional positional deviation of the tumor at the N^{th} moment with the upper limit of the predetermined deviation range to determine whether the square root of the three-dimensional positional deviation of the tumor at the N^{th} moment is greater than the upper limit of the predetermined deviation range. For the process that the processing component calculates the square root of the three-dimensional positional deviation of the tumor at the N^{th} moment, reference may be made to sub-step 3041, which is not repeated here in this embodiment.

In step 3043, the position of the tumor does not need to be corrected if the three-dimensional positional deviation of the tumor at the N^{th} moment is smaller than a lower limit of the predetermined deviation range.

The control component may compare the three-dimensional positional deviation of the tumor at the N^{th} moment with the lower limit of the predetermined deviation range to determine whether the three-dimensional positional deviation of the tumor at the N^{th} moment is smaller than the lower limit of the predetermined deviation range. If the three-dimensional positional deviation of the tumor at the N^{th} moment is smaller than the lower limit of the predetermined deviation range, it indicates that the positional deviation of the tumor is relatively small, or the tumor has no positional deviation. In this case, the position of the tumor does not need to be corrected. For example, it is unnecessary to move the treatment bed, to adjust the multi-leaf collimator, to perform the alarm operation, and the like.

Optionally, the predetermined deviation range may be a deviation range of the square root of the three-dimensional positional deviation. The control component may calculate the square root of the three-dimensional positional deviation of the tumor at the N^{th} moment according to the square root formula, and then compare the square root of the three-dimensional positional deviation of the tumor at the N^{th} moment with the lower limit of the predetermined deviation range to determine whether the square root of the three-dimensional positional deviation of the tumor at the N^{th} moment is smaller than the lower limit of the predetermined deviation range. For the process that the processing component calculates the square root of the three-dimensional positional deviation of the tumor at the N^{th} moment, reference may be made to sub-step 3041, which is not repeated here in this embodiment.

In summary, the tumor tracking method according to this embodiment of the present disclosure includes: acquiring the tumor image at the N^{th} moment; determining the two-dimensional positional deviation of the tumor at the N^{th} moment according to the tumor image at the N^{th} moment and the tumor reference image corresponding to the tumor image at the N^{th} moment; determining the three-dimensional positional deviation of the tumor at the N^{th} moment according to the two-dimensional positional deviation of the tumor at the N^{th} moment and the predetermined two-dimensional positional deviation of the tumor at the (N−1)^{th} moment; and tracking the tumor according to the three-dimensional positional deviation of the tumor at the N^{th} moment. Since the tumor tracking may be achieved just by adopting the tumor image acquisition apparatus (that is, an imaging system, which includes an imaging source and a detector), the present disclosure solves the problem that a tumor tracking device is highly complex and costly, and contributes to reducing the complexity and the cost of the tumor tracking device.

FIG. 6 is a flowchart of another tumor tracking method according to an embodiment of the present disclosure. This embodiment is described by using the scenario where the tumor tracking method is applied to the radiotherapy device as shown in FIG. 1 as an example. The tumor tracking method may be performed by a tumor tracking apparatus. The tumor tracking apparatus includes the tumor image acquisition apparatus (including the imaging source and the detector), the processing component, and the control component in the radiotherapy device as shown in FIG. 1. Referring to FIG. 6, the tumor tracking method includes the following steps:

In step 601, a first image of a tumor is acquired at a first moment.

The tumor image acquisition apparatus may acquire the first image of the tumor at the first moment. The tumor image acquisition apparatus may include an imaging source and a detector, and may circumferentially rotate about the tumor. At the first moment, the imaging source may emit rays, the rays pass through the tumor, reach the detector, and are received by the detector. The processing component determines an image of the tumor according to the rays received by the detector, that is, the first image of the tumor acquired at the first moment. The rays may be X-rays.

Exemplarily, as shown in FIG. 2, it is assumed that the first imaging source 02 rotates to the position point A1, then the imaging source 02 emits the rays from the position point A1 to the tumor in the body of the patient 07. The rays pass through the body of the patient 07, reach the detector 03 and are received by the detector 03. The processing component determines an image of the tumor according to the rays received by the detector 03, that is, the first image of the tumor acquired at the first moment.

In step 602, a second image of the tumor is acquired at a second moment.

The first moment and the second moment are adjacent moments, and the first image and the second image have different image planes. Optionally, an included angle between the image plane of the first image and the image plane of the second image is within a value range of 45-135 degrees. For example, the included angle between the image plane of the first image and the image plane of the second image is 90 degrees.

The process that the tumor image acquisition apparatus acquires the second image of the tumor at the second moment may be referenced to step 601. Exemplarily, as shown in FIG. 2, it is assumed that the second imaging source 02 rotates to the position point A2, then the imaging source 02 emits rays from the position point A2 to the tumor in the body of the patient 07. The rays pass through the body of the patient 07, reach the detector 03, and are received by the detector 03. The processing component determines an image of the tumor according to the rays received by the detector, that is, the second image of the tumor acquired at the second moment.

It should be noted that the image plane of the first image may refer to a projection plane of the rays emitted by the imaging source to the tumor at the first moment, for example, a projection plane of the rays emitted by the imaging source from the position point A1 to the tumor in the body of the patient 07; and the image plane of the second image may refer to a projection plane of the rays emitted by the imaging source to the tumor at the second moment, for example, a projection plane of the rays emitted by the imaging source from the position A2 to the tumor in the body of the patient 07.

In step 603, a position of the tumor is determined based on the first image and the second image.

Optionally, the tumor image acquisition apparatus may determine a two-dimensional positional deviation of the tumor at the first moment according to the first image and a tumor reference image (that is, a tumor reference image corresponding to the position point of the imaging source at the first moment) corresponding to the first image, determine a two-dimensional positional deviation of the tumor at the second moment according to the second image and a tumor reference image (that is, a tumor reference image corresponding to the position point of the imaging source at the second moment) corresponding to the second image, determines a three-dimensional positional deviation of the tumor at the second moment according to the two-dimensional positional deviation of the tumor at the second moment and the two-dimensional positional deviation of the tumor at the first moment, and determine the position of the tumor according to the three-dimensional positional deviation of the tumor at the second moment, that is, tracking the tumor according to the three-dimensional positional deviation of the tumor at the second moment.

In summary, in the tumor tracking method according to this embodiment of the present disclosure, after the first tumor image is acquired at the first moment and the second image of the tumor is acquired at the second moment, the position of the tumor is determined according to the first image and the second image such that the tumor is tracked. Since the tumor may be tracked by using only the tumor image acquisition apparatus (that is, one imaging system is employed, wherein this imaging system includes an imaging source and a detector), the problems of high complexity of the tumor tracking device and high cost of the device are addressed, which facilitates reduction of the complexity of the tumor tracking device and the cost of the device.

Optionally, FIG. 7 is a flowchart of still another tumor tracking method according to an embodiment of the present disclosure. Referring to FIG. 7, based on FIG. 6, the tumor tracking method further includes the following steps:

In step 604, a third image of the tumor is acquired at a third moment.

The second moment and the third moment are adjacent moments, and the second image and the third image have different image planes. An included angle between the image plane of the second image and the image plane of the third image is within the value range of 45-135 degrees. Optionally, a time interval between the first moment and the second moment is equal to or not equal to a time interval between the second moment and the third moment. The implementation of step 604 may be referenced to step 601, which is not described herein any further.

In step 605, the position of the tumor is determined according to the second image and the third image.

The implementation of step 605 may be referenced to step 603, which is not described herein any further.

In the tumor tracking method according to this embodiment of the present disclosure, after the second image of the tumor is acquired at the second moment and the third image of the tumor is acquired at the third moment, the position of the tumor is determined according to the second image and the third image such that the tumor is tracked. Since the tumor may be tracked by using only the tumor image acquisition apparatus (that is, one imaging system is employed, wherein this imaging system includes an imaging source and a detector), the problems of high complexity of the tumor tracking device and high cost of the device are addressed, which facilitates reduction of the complexity of the tumor tracking device and the cost of the device. At the same time, the tumor images obtained at adjacent moments are used to track the position of the tumor, which improves the efficiency of tumor tracking.

According to the tumor tracking method according to this embodiment of the present disclosure, during the entire tumor tracking process, the rotation frame rotates to drive the tumor image acquisition apparatus (including an imaging source and a detector) to rotate circumferentially. During the rotation of the tumor image acquisition apparatus, the tumor image acquisition apparatus may acquire tumor images at regular intervals. The tumor tracking apparatus may determine the two-dimensional positional deviations of the tumor at different moments according to the tumor images at different moments, and then calculate a three-dimensional deviation of the tumor in conjunction with the two-dimensional positional deviations of the tumor acquired for two adjacent times. The tumor tracking apparatus may respond to the movement of the tumor in real time according to the three-dimensional deviation of the tumor, thereby achieving real-time tracking of the tumor during the entire treatment process. In this embodiment of the present disclosure, a time interval between any two adjacent moments may be equal or unequal. In addition, the time interval between any two adjacent moments may be adjusted. The any two adjacent moments refer to moments at which the tumor image acquisition apparatus acquires the tumor images for any two adjacent times.

It should be noted that, in practice, if the tumor image acquisition apparatus acquires the tumor images for two consecutive times, an included angle between position points at which the imaging source is located is relatively small, such that the error of the finally determined three-dimensional positional deviation of the tumor is relatively large. Therefore, in this embodiment, the tumor tracking apparatus may adjust the time interval between any two adjacent moments to increase the included angle between the position points of the imaging source at the moments at which the tumor images are acquired for any two adjacent times, thereby reducing the error of the determined three-dimensional positional deviation of the tumor and improving the accuracy of tumor tracking.

The tumor tracking method according to the embodiment shown in FIGS. 3 to 7 may be applied to a radiotherapy device. The radiotherapy device includes a rotation frame, and a treatment head and a tumor image acquisition apparatus which are provided on the rotation frame. The rotation frame drives the treatment head and the tumor image acquisition apparatus to rotate around the tumor. The schematic diagram of the radiotherapy device may be referenced to FIG. 1.

The tumor tracking method according to the embodiment shown in FIGS. 3 to 7 may be applied in a rotating treatment mode, a drawn-arc treatment mode and a fixed-point treatment mode. In the rotating treatment mode, the rotation frame drives the treatment head and the tumor image acquisition apparatus to rotate around the circumference of the tumor. The drawn-arc treatment mode may be a large-angle or small-angle drawn-arc treatment mode. In the drawn-arc treatment mode, during the treatment, the rotation frame rotates (which may rotate at a uniform speed or a non-uniform speed) to drive the treatment head to move in a drawn-arc segment. In the fixed-point treatment mode, during the treatment, the treatment head stays at a certain point to treat the tumor.

In the drawn-arc treatment mode, especially in the case of small-angle drawn-arc treatment, owing to a small drawn-arc angle, when the tumor image acquisition apparatus acquires the tumor images for two consecutive times, an included angle between the position points at which the imaging source is located is relatively small, such that the error of the finally determined three-dimensional positional deviation of the tumor is relatively large. For improvement of the tumor tracking precision in the drawn-arc treatment process, in this embodiment of the present disclosure, a virtual treatment point is arranged outside the arc segment. In the treatment process, the treatment head may also move outside the arc segment. The treatment head is positioned at the virtual treatment point at the N^{th} moment or the (N−1)^{th} moment; and an arc segment between a position point at which the treatment head is located at the N^{th} moment and a position point at which the treatment head is located at the (N−1)^{th} moment is greater than the drawn-arc segment.

In this embodiment of the present disclosure, in the drawn-arc treatment mode, the tumor image acquisition apparatus may acquire tumor images. In addition, at the moments at which the tumor images are acquired for two consecutive times, the treatment head at one moment may be located in the arc segment, and the treatment head at the other moment may be located at a virtual treatment point outside the arc segment. Alternatively, at the moments at which the tumor images are acquired for two consecutive times, the treatment head is located at two different virtual treatment points outside the arc segment. Since the included angle between the treatment head and the tumor image acquisition apparatus is fixed, the included angle between the position points at which the tumor image acquisition apparatus is located is relatively large if the included angle between the position points of the treatment head at the moments at which the tumor images are acquired for two consecutive times is relatively large.

Exemplarily, referring to FIG. 8, a schematic diagram in which tumor images are acquired in the drawn-arc treatment mode according to an embodiment of the present disclosure is illustrated. Referring to FIG. 8, the arc segment may be a circular arc between a position point E1 and a position point E2. Any position point on the circular arc may be a treatment point, and the position points E1 and E2 may also be treatment points. The treatment head may move within the arc segment and emit radioactive rays. According to the tumor tracking apparatus, a virtual treatment point E3 may be provided outside the drawn-arc segment. During the treatment, the treatment head may also move outside the arc segment. When the treatment head moves outside the arc segment, the treatment head does not emit radioactive rays. In this embodiment of the present disclosure, at the (N−1)^{th} moment, the treatment head may be located at a position point E4 within the arc segment. At the N^{th} moment, the treatment head may be located at a virtual treatment point E3 outside the arc segment. An arc segment between the position point at which the treatment head is located at the (N−1)^{th} moment and the position point at which the treatment head is located at the N^{th} moment is greater than the drawn-arc segment, such that the arc segment between the position point at which the tumor image acquisition apparatus is located at the (N−1)^{th} moment and the position point at which the tumor image acquisition apparatus is located at the N^{th} moment is greater than the drawn-arc segment. At the (N−1)^{th} moment and the N^{th} moment, the tumor image acquisition apparatus acquires tumor images once respectively. In addition, the tumor image acquired by the tumor image acquisition apparatus at the (N−1)^{th} moment is the tumor image at the (N−1)^{th} moment, and the tumor image acquired by the tumor image acquisition apparatus at the N^{th} moment is the tumor image at the N^{th} moment.

It should be noted that this embodiment of the present disclosure is illustrated by taking a case in which the treatment head at the (N−1)^{th} moment is located in the drawn-arc segment and the treatment head at the N^{th} moment is located at the virtual treatment point as an example. In practice, the treatment head at the (N−1)^{th} moment may be located at the virtual treatment point, and the treatment head at the N^{th} moment may be located within the drawn-arc segment. In the case that at least two virtual treatment points are provided outside the drawn-arc segment, the treatment head at the (N−1)^{th} moment and the treatment head at the N^{th} moment may be located at two different virtual treatment points among at least two virtual treatment points, as long as it is ensured that the arc segment between the position point at which the treatment head is located at the N^{th} moment and the position point at which the treatment head is located at the (N−1)^{th} moment is greater than the drawn-arc segment.

In this embodiment, by providing the virtual treatment points, the tumor image acquisition apparatus may acquire the tumor images when the treatment heads are located within the drawn-arc segment and at the virtual treatment point, or when the treatment heads are located at two different virtual treatment points. In this way, when the tumor image acquisition apparatus acquires the tumor images for two adjacent times, the included angle between the position points at which the tumor image acquisition apparatus is located increases, and an error of the finally determined three-dimensional positional deviation of the tumor is reduced, thereby improving the accuracy of tumor tracking.

After the tumor images are acquired, the methods for determining the three-dimensional positional deviation according to the acquired tumor images and tracking the tumor are as described above, and are not repeated herein.

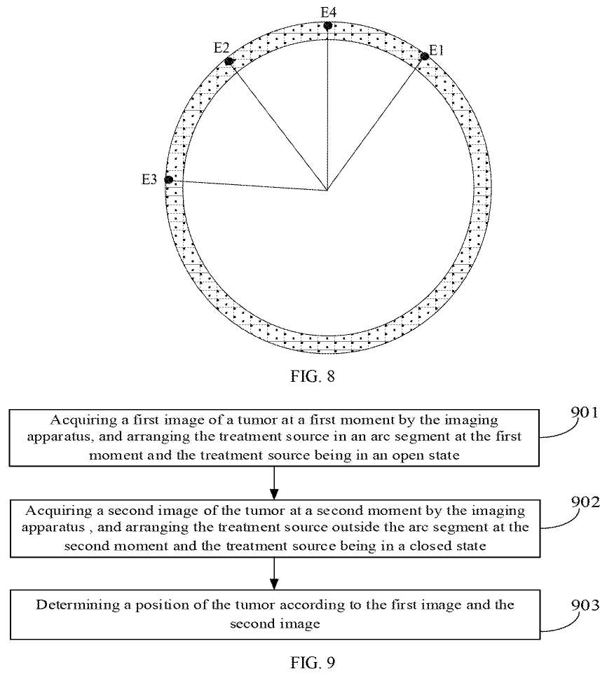

Exemplarily, FIG. 9 is a flowchart of a method for tracking the tumor in an arc treatment mode according to an embodiment of the present disclosure. The method may be applied to a radiotherapy device. The radiotherapy device includes a rotation frame, a treatment source, and an imaging apparatus (that is, the tumor image acquisition apparatus as described above). The treatment source and the imaging apparatus are both fixedly arranged on the rotation frame, and the treatment source and the imaging apparatus rotate about the tumor under driving by the rotation frame. Referring to FIG. 9, the tumor tracking method may include the following steps:

In step 901, the imaging apparatus acquires a first image of a tumor at a first moment, and the treatment source is arranged in an arc segment at the first moment and is in an open state.

Optionally, the imaging apparatus may include an imaging source and a detector. The imaging apparatus is capable of circumferentially rotating about the tumor. At the first moment, the imaging source may emit rays to the tumor. The rays pass through the tumor, reach the detector and are received by the detector. The processing component determines an image of the tumor according to the rays received by the detector, that is, the first image of the tumor acquired by the imaging apparatus at the first moment.

Optionally, in the arc treatment mode, at the first moment, the treatment source may be arranged in the arc segment and in the open state. That is, at the first moment, the treatment source is arranged in the arc segment and emits rays to the tumor for treatment of the tumor. Exemplarily, as shown in FIG. 8, at the first moment, the treatment head may be arranged at a position point E4 in the arc segment (an arc formed between a position point E1 and a position point E2), and emits rays to the tumor.

In step 902, the imaging apparatus acquires a second image of the tumor at a second moment, and the treatment source is arranged outside the arc segment at the second moment and is in a closed state.

Optionally, the imaging apparatus may include the imaging source and the detector. The imaging apparatus is capable of circumferentially rotating about the tumor. At the second moment, the imaging apparatus may emit rays to the tumor. The rays pass through the tumor, reach the detector, and are received by the detector. The processing component determines an image of the tumor according to the rays received by the detector, that is, the second image of the tumor acquired by the imaging apparatus at the first moment.

Optionally, in the arc treatment mode, at the second moment, the treatment source may be arranged outside the arc segment and in the closed state. That is, at the second moment, the treatment source is arranged outside the arc segment and does not emit rays. Exemplarily, as shown in FIG. 8, at the second moment, the treatment head may be arranged at a position point outside the arc segment (the arc formed between the position point E1 and the position point E2), and does not emit rays.

In this embodiment of the present disclosure, the first moment and the second moment may be adjacent moments, an included angle between a position at which the treatment source is located at the first moment and a position at which the treatment is located at the second moment is within a value range of 45-135 degrees. For example, the included angle between the position at which the treatment source is located at the first moment and the position at which the treatment is located at the second moment may be 90 degrees. Exemplarily, as shown in FIG. 8, an included angle between the position point E3 and the position point E4 may be 90 degrees.

In step 903, a position of the tumor is determined according to the first image and the second image.

The implementation of step 903 may be referenced to step 602, which is not described herein any further.

In summary, in the method for tracking the tumor in the arc treatment mode according to this embodiment of the present disclosure, since the treatment source is arranged in the arc segment at the first moment and arranged outside the arc segment at the second moment, the included angle between the position points at which the treatment head is located at the first moment and the second moment may be made relatively greater. In the radiotherapy device, an included angle between the treatment head and the imaging apparatus is fixed. If the included angle between the position points at which the treatment head is located at the first moment and the second moment is relatively greater, an included angle between the position points at which the imaging apparatus is located at the first moment and the second moment is also greater. Therefore, an error of a three-dimensional positional deviation of the tumor determined based on the first image acquired by the imaging apparatus at the first moment and the second image acquired by the imaging apparatus at the second moment is smaller, which facilitates improvement of the precision in tracking the tumor during the arc treatment. For the tumor tracking method shown in FIG. 3 to FIG. 7 in the fixed-point treatment mode, in this embodiment of the present disclosure, a virtual treatment point is provided outside a fixed point. During the treatment, the treatment head is moveable outside the fixed point. The treatment head is located at the virtual treatment point at the N^{th} moment or the (N−1)^{th} moment.

In this embodiment of the present disclosure, in the fixed-point treatment mode, the tumor image acquisition apparatus may acquire tumor images. In addition, at the moments at which the tumor images are acquired for two consecutive times, the treatment head at one moment may be located at a fixed point (which refers to a treatment point at which the treatment head emit radioactive rays), and the treatment head at the other moment may be located at a virtual treatment point outside the fixed point (the treatment head at the virtual treatment point does not emit radioactive rays). Alternatively, at the moments at which the tumor images are acquired for two consecutive times, the treatment heads are located at two different virtual treatment points outside the fixed point. In this way, the tumor tracking method shown in FIGS. 3 to 7 may be applied to the fixed-point treatment process.

Since the included angle between the treatment head and the tumor image acquisition apparatus is fixed, the included angle between the position points at which the tumor image acquisition apparatus is located is relatively large if the included angle between the position points of the treatment head at the moments at which the tumor images are acquired for two consecutive times is relatively large. For improvement of the accuracy of tumor tracking during the fixed-point treatment, a value range of the included angle between the position points of the treatment head at the moments at which the tumor images are acquired for two consecutive times may be 45-135 degrees, and preferably 90 degrees.

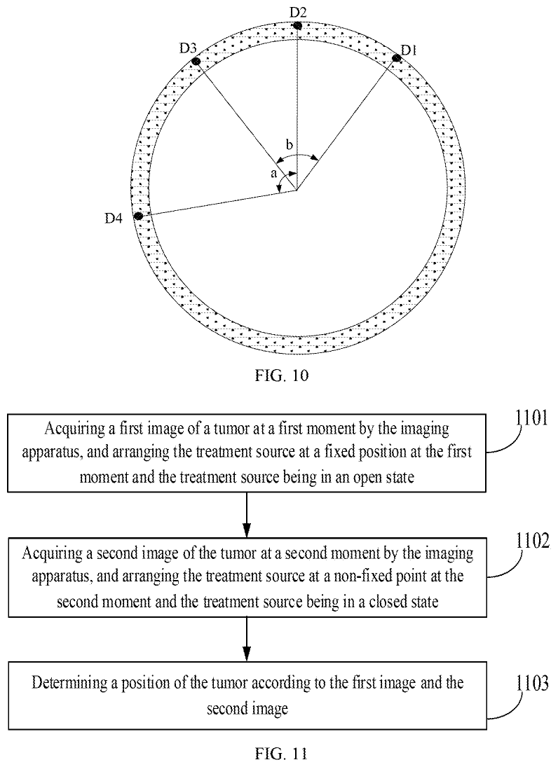

FIG. 10 is a schematic diagram in which the tumor images are acquired in a fixed-point treatment mode according to an embodiment of the present disclosure. Referring to FIG. 10, a fixed point D2 is a treatment point, and the treatment head may emit radioactive rays at the fixed point D2. According to the tumor tracking apparatus, virtual treatment points D1, D3, and D4 may be provided outside the fixed point D2. During the treatment, the treatment head may also move outside the fixed point D2. When the treatment head moves outside the fixed point D2, the treatment head does not emit radioactive rays. For example, when the treatment head is located at the virtual treatment points D1, D3 or D4, none of the treatment heads emits radioactive rays. In this embodiment of the present disclosure, at the (N−1)^{th} moment, the treatment head may be located at the fixed point D2. At the N^{th} moment, the treatment head may be located at the virtual treatment point D4. An included angle between the fixed point D2 and the virtual treatment point D4 may be a, and a value range of the included angle a may be 45-135 degrees. Therefore, the value range of the included angle between the position point at which the tumor image acquisition apparatus is located at the N^{th} moment and the position point at which the tumor image acquisition apparatus is located at the (N−1)^{th} moment may be 45-135 degrees. Alternatively, in this embodiment of the present disclosure, at the (N−1)^{th} moment, the treatment head may be located at the virtual treatment point D1. At the N^{th} moment, the treatment head may be located at the virtual treatment point D3. An included angle between the virtual treatment point D1 and the virtual treatment point D3 may be b, and a value range of the included angle b may be 45-135 degrees. Therefore, the value range of the included angle between the position point at which the tumor image acquisition apparatus is located at the N^{th} moment and the position point at which the tumor image acquisition apparatus is located at the (N−1)^{th} moment may be 45-135 degrees.

It should be noted that, in practice, FIG. 10 is merely exemplary. In practice, the treatment head at the (N−1)^{th} moment may be located at the virtual treatment point, and the treatment head at the N^{th} moment may be located at the fixed point. Alternatively, the treatment head at the (N−1)^{th} moment and the treatment head the N^{th} moment are located at different virtual treatment points, as long as it is ensured that the position point at which the treatment head is located at the N^{th} moment is different from the position point at which the treatment head is located at the (N−1)^{th} moment.

In this embodiment, by providing the virtual treatment points, the treatment head does not emit radioactive rays while rotating to the virtual treatment point. However, the tumor image acquisition apparatus may acquire tumor images. In addition, the tumor image acquisition apparatus may acquire the tumor images when the treatment heads are located at the fixed point and at the virtual treatment point. In this way, when the tumor image acquisition apparatus acquires the tumor images for two adjacent times, the position points at which the tumor image acquisition apparatus is located are different, thereby achieving the application of the tumor tracking method described in FIGS. 3 to 7 in the fixed-point treatment mode. When the tumor image acquisition apparatus acquires the tumor images for two consecutive times, the value range of the included angle between the position points at which the tumor image acquisition apparatus is located is 45-135 degrees, and the error of the finally determined three-dimensional positional deviation of the tumor is reduced, thereby improving the accuracy of tumor tracking.

After the tumor images are acquired, the methods for determining the three-dimensional positional deviation according to the acquired tumor images and tracking the tumor are as described above, and are not repeated here.

Exemplarily, FIG. 11 is a flowchart of a method for tracking the tumor in the fixed-point treatment mode according to an embodiment of the present disclosure. The method may be applied to a radiotherapy device. The radiotherapy device includes a rotation frame, a treatment source, and an imaging system (that is, the tumor imaging acquisition apparatus as described above). The treatment source and the imaging apparatus are both fixedly arranged on the rotation frame, and the treatment source and the imaging apparatus rotate about the tumor under driving by the rotation frame. Referring to FIG. 11, the tumor tracking method may include the following steps:

In step 1101, the imaging apparatus acquires a first image of a tumor at a first moment, and the treatment source is arranged at a fixed position at the first moment and is in an open state.

Optionally, the imaging apparatus may include an imaging source and a detector. The imaging apparatus is capable of circumferentially rotating about the tumor. At the first moment, the imaging source may emit rays to the tumor. The rays pass through the tumor, reach the detector and are received by the detector. The processing component determines an image of the tumor according to the rays received by the detector, that is, the first image of the tumor acquired by the imaging apparatus at the first moment.

Optionally, in the fixed-point treatment mode, at the first moment, the treatment source may be arranged at the fixed point and in the open state. That is, at the first moment, the treatment source is arranged at fixed point and emits rays to the tumor for treatment of the tumor. Exemplarily, as shown in FIG. 10, at the first moment, the treatment head may be arranged at a position of a fixed point D2, and emits rays to the tumor.

In step 1102, the imaging apparatus acquires a second image of the tumor at a second moment, and the treatment source is arranged at a non-fixed point at the second moment and is in a closed state.

Optionally, the imaging apparatus may include the imaging source and the detector. The imaging apparatus is capable of circumferentially rotating about the tumor. At the second moment, the imaging apparatus may emit rays to the tumor. The rays pass through the tumor, reach the detector, and are received by the detector. The processing component determines an image of the tumor according to the rays received by the detector, that is, the second image of the tumor acquired by the imaging apparatus at the first moment.

Optionally, in the fixed-point treatment mode, at the second moment, the treatment source may be arranged at the non-fixed point and in the closed state. That is, at the second moment, the treatment source is arranged at the non-fixed point and does not emit rays. Exemplarily, as shown in FIG. 10, at the second moment, the treatment head may be arranged at a position of a virtual treatment point D4, and does not emit rays.

In this embodiment of the present disclosure, the first moment and the second moment may be adjacent moments, an included angle between a position at which the treatment source is located at the first moment and a position at which the treatment is located at the second moment is within a value range of 45-135 degrees. For example, the included angle between the position at which the treatment source is located at the first moment and the position at which the treatment is located at the second moment may be 90 degrees. Exemplarily, as shown in FIG. 10, an included angle between the fixed point D2 and the virtual treatment point D4 may be 90 degrees.

In step 1103, a position of the tumor is determined according to the first image and the second image.

The implementation of step 1103 may be referenced to step 603, which is not described herein any further.

In summary, in the method for tracking the tumor in the fixed-point treatment mode according to this embodiment of the present disclosure, since the treatment source is arranged at the fixed point at the first moment and arranged at the non-fixed point at the second moment, the included angle between the position points at which the treatment head is located at the first moment and the second moment may be made relatively greater. In the radiotherapy device, an included angle between the treatment head and the imaging apparatus is fixed. If the included angle between the position points at which the treatment head is located at the first moment and the second moment is relatively greater, an included angle between the position points at which the imaging apparatus is located at the first moment and the second moment is also greater. Therefore, an error of a three-dimensional positional deviation of the tumor determined based on the first image acquired by the imaging apparatus at the first moment and the second image acquired by the imaging apparatus at the second moment is smaller, which facilitates improvement of the precision in tracking the tumor during the arc treatment.

An imaging system in the tumor tracking apparatus of the present disclosure may be configured for image guidance before the treatment, and may also be configured for tumor tracking during the treatment, such that the utilization rate of the imaging system (including an imaging source and a detector) is relatively high. The image guidance before treatment is used to accurately position the tumor before the treatment. The image guidance before the treatment is used to make the tumor coincide with a treatment isocenter of the radiotherapy device. The image guidance may specifically include: acquiring tumor images at a first position and a second position by using the imaging system respectively to obtain two tumor images; comparing each of the two tumor images with a tumor reference image at the corresponding position to obtain two two-dimensional positional deviations of the tumor; calculating a three-dimensional positional deviation of the tumor according to the two two-dimensional positional deviations; and correcting the position of the tumor according to the three-dimensional positional deviation, such that the tumor coincides with the treatment isocenter of the radiotherapy device.

The radiotherapy system according to this embodiment of the present disclosure may be configured for image guidance before the treatment, and may also be configured for tumor tracking during the treatment. In terms of the image guidance before the treatment, the imaging system only needs to be exposed at a predetermined position point. However, in terms of the tumor tracking during the treatment, the imaging system needs to be continuously exposed at certain time intervals. Therefore, for cooperation between the image guidance before the treatment with the tumor tracking during the treatment without affecting each other, an embodiment of the present disclosure further provides a radiotherapy system.

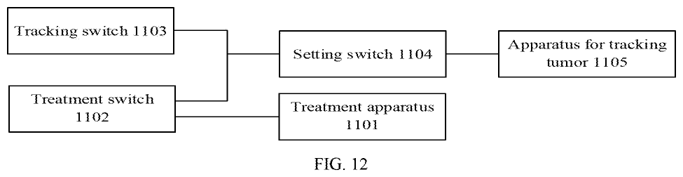

FIG. 12 is a schematic structural diagram of a radiotherapy system according to an embodiment of the present disclosure. Referring to FIG. 12, the radiotherapy system includes a treatment apparatus 1101, a treatment switch 1102, a tracking switch 1103, a setting switch 1104, and a tumor tracking device 1105. The tumor tracking apparatus 1105 may be the tumor tracking apparatus 100 shown in FIG. 8. The treatment switch 1102 is connected in parallel to the tracking switch 1103. The setting switch 1104 is connected in series to the treatment switch 1102 and the tracking switch 1103, respectively. The treatment apparatus 1101 is connected to the treatment switch 1102. The tumor tracking apparatus 1105 is connected to the setting switch 1104.

The tracking switch 1103 may also be referred to as an exposure switch, and the setting switch 1104 may also be referred as to a software setting exposure switch. The tracking switch 1103 is configured to control the imaging system of the radiotherapy system to perform image guidance before the treatment. The treatment switch 1102 is configured to control the treatment head of the radiotherapy system to treat the tumor, and to control the imaging system of the radiotherapy system to track the tumor during the treatment. The setting switch 1104 is configured to control the imaging system of the radiotherapy system to switch between the image guidance before the treatment and the tumor tracking during the treatment.

The control process of the radiotherapy system according to the embodiment of the present disclosure may be as follows:

in the case of the image guidance before the treatment, the tracking switch 1103 and the setting switch 1104 are turned on at the same time; the setting switch 1104 is selected for the image guidance before the treatment; the treatment switch 1102 is turned off; and the tumor tracking apparatus 1105 operates to achieve the image guidance before the treatment.

During the treatment, in the case that the tumor tracking is not required, the treatment switch 1102 is turned on, the tracking switch 1103 and the setting switch 1104 are turned off, and the treatment apparatus 1101 operates to achieve the treatment of the tumor. In the case that the tumor tracking is required, the treatment switch 1102 and the setting switch 1104 are turned on at the same time, the setting switch 1104 is selected for the tumor tracking during the treatment; the tracking switch 1103 is turned off; and the tumor tracking apparatus 1105 and the treatment apparatus 1101 operate simultaneously to achieve the tumor tracking during the treatment.

In summary, the radiotherapy system according to the present disclosure is configured to acquire the tumor image at the N^{th} moment; determine the two-dimensional positional deviation of the tumor at the N^{th} moment according to the tumor image at the N^{th} moment and the tumor reference image corresponding to the tumor image at the N^{th} moment; determine the three-dimensional positional deviation of the tumor at the N^{th} moment according to the two-dimensional positional deviation of the tumor at the N^{th} moment and the predetermined two-dimensional positional deviation of the tumor at the (N−1)^{th} moment; and track the tumor according to the three-dimensional positional deviation of the tumor at the N^{th} moment. Since the tumor tracking may be achieved just by adopting the tumor image acquisition apparatus (that is, an imaging system, which includes an imaging source and a detector), the present disclosure solves the problem that a tumor tracking device is highly complex and costly, and contributes to reducing the complexity and the cost of the tumor tracking device.

According to the radiotherapy system according to the embodiment of the present disclosure, with the treatment switch, the tracking switch and the setting switch, the radiotherapy system may not only achieve the image guidance before the treatment, but also track the tumor during the treatment, but also achieve individual treatment, all of which operate cooperatively without affecting with each other, thereby improving the utilization rate of the imaging system.

Based on the same inventive concept, an embodiment of the present disclosure further provides a tumor tracking apparatus. The tumor tracking apparatus includes a processor. The processor is configured to:

acquire a first image of a tumor at a first moment;

acquire a second image of the tumor at a second moment; and

determine a position of the tumor based on the first image and the second image;

wherein the first moment and the second moment are adjacent moments, and the first image and the second image have different image planes.

Optionally, the processor is further configured to:

acquire a third image of the tumor at a third moment; and

determine the position of the tumor based on the second image and the third image;

wherein the second moment and the third moment are adjacent moments, and the second image and the third image have different image planes.

Optionally, a time interval between the first moment and the second moment is equal to or not equal to a time interval between the second moment and the third moment.

An included angle between the image plane of the first image and the image plane of the second image is within the value range of 45-135 degrees and an included angle between the image plane of the second image and the image plane of the third image is within the value range of 45-135 degrees.

An embodiment of the present disclosure further provides a computer readable storage medium configured to store at least one instruction therein, which, when running on a processing component of a computer, causes the processing component to perform the method as shown in any one of FIGS. 3 to 5.

An embodiment of the present disclosure further provides a computer program product, which, when running on a computer, causes the computer to perform the method as shown in any one of FIGS. 3 to 5.

It should be noted that the tumor tracking solutions according to the embodiments of the present disclosure may implement tumor tracking by using only one imaging system. However, it may be understood that the tumor tracking solutions may also be applicable to at least two imaging systems for tumor tracking, which are not limited in the embodiments of the present disclosure.

It should be noted that for a radiotherapy apparatus implementing the tumor tracking method described in the embodiments of the present disclosure, when it has only one imaging system, the imaging system should be able to rotate, while the treatment head can be rotated or not; when it has at least two sets of imaging systems, the two sets of imaging systems may be fixedly set or may be rotated, Similarly, the treatment head can be rotated or not, and which are not limited in the embodiments of the present disclosure.

Persons of ordinary skill in the art may understand that all or part of the steps described in the above embodiments may be performed by hardware, or by relevant hardware instructed by applications stored in a computer readable storage medium, such as a read-only memory, a disk, a CD, or the like.

Described above are merely exemplary embodiments of the present disclosure, and are not intended to limit the present disclosure. Within the spirit and principles of the disclosure, any modifications, equivalent substitutions, improvements, or the like are within the protection scope of the present disclosure.

1. A tumor tracking method, which is applied to a radiotherapy device comprising a tumor image acquisition apparatus and configured to acquire tumor images at different moments, the method comprising:
acquiring a tumor image at an N^{th} moment, wherein N=2, 3, 4 . . . M, M being a positive integer;
determining a two-dimensional positional deviation of the tumor at the N^{th} moment according to the tumor image at the N^{th} moment and a tumor reference image corresponding to the tumor image at the N^{th} moment;
determining a three-dimensional positional deviation of the tumor at the N^{th} moment according to the two-dimensional positional deviation of the tumor at the N^{th} moment and a predetermined two-dimensional positional deviation of the tumor at an (N−1)^{th} moment, wherein the two-dimensional positional deviation of the tumor at the (N−1)^{th} moment is a two-dimensional positional deviation determined according to a tumor image at the (N−1)^{th} moment and a tumor reference image corresponding to the tumor image at the (N−1)^{th} moment, and a position at which the tumor image at the N^{th} moment is acquired is different from a position at which the tumor image at the (N−1)^{th} moment is acquired; and
tracking the tumor according to the three-dimensional positional deviation of the tumor at the N^{th} moment.

2. The tumor tracking method according to claim 1, wherein time intervals between any two adjacent moments are equal or unequal.

3. The tumor tracking method according to claim 1, wherein an included angle between the position at which the tumor image at the N^{th} moment is acquired and the position at which the tumor image at the (N−1)^{th} moment is acquired is within a value range of 45-135 degrees.

4. The tumor tracking method according to claim 1, wherein tracking the tumor according to the three-dimensional positional deviation of the tumor at the N^{th} moment comprises:
tracking the tumor according to a relationship between the three-dimensional positional deviation of the tumor at the N^{th} moment and a predetermined deviation range.

5. A tumor tracking method, comprising:
acquiring a first image of a tumor at a first moment;
acquiring a second image of the tumor at a second moment; and
determining a position of the tumor based on the first image and the second image;
wherein the first moment and the second moment are adjacent moments, and the first image and the second image have different image planes.

6. The method according to claim 5, further comprising:
acquiring a third image of the tumor at a third moment; and
determining the position of the tumor based on the second image and the third image;
wherein the second moment and the third moment are adjacent moments, and the second image and the third image have different image planes.

7. The method according to claim 6, wherein a time interval between the first moment and the second moment is equal to or not equal to a time interval between the second moment and the third moment.

8. The method according to claim 6, wherein an included angle between the image plane of the first image and the image plane of the second image is within a value range of 45-135 degrees, and an included angle between the image plane of the second image and the image plane of the third image is in the value range of 45-135 degrees.

9. A tumor tracking apparatus, comprising a processor, configured to:
acquire a first image of a tumor at a first moment;
acquire a second image of the tumor at a second moment; and
determine a position of the tumor based on the first image and the second image;
wherein the first moment and the second moment are adjacent moments, and the first image and the second image have different image planes.

10. The tumor tracking apparatus according to claim 9, wherein the processor is further configured to:
acquire a third image of the tumor at a third moment; and
determine the position of the tumor based on the second image and the third image;
wherein the second moment and the third moment are adjacent moments, and the second image and the third image have different image planes.
